(19) United States
(12) Patent Application Publication
(10) Pub. No.: US 2012/0101478 A1
(43) Pub. Date: Apr. 26, 2012

(54) Dual Cartridge Mixer Syringe

(75) Inventors: Dimitrios Stroumpoulis, Goleta, CA (US); Edwin J. Kayda, Santa Barbara, CA (US)

(73) Assignee: ALLERGAN, INC., Irvine, CA (US)

(21) Appl. No.: 12/909,216

(22) Filed: Oct. 21, 2010

(51) Int. Cl. A61M 5/31

(52) U.S. Cl. 604/518; 604/82

(57) ABSTRACT

A dual cartridge mixer syringe includes a first cartridge for containing a first formulation, a second cartridge for containing a second formulation, a first plunger for displacing said first formulation from said first cartridge, and a second plunger for displacing said second formulation from said second cartridge. A mixing assembly is provided for receiving and commingling the first and second formulation in unequal proportions; and a needle assembly, in fluid communication with said mixing assembly, provides for ejecting the commingled first and second formulation.

The present invention generally relates to syringe systems and is more particularly directed to syringe systems for mixing and dispensing two fluids stored in individual compartments.

The present invention is useful in the administration of gels that have cosmetic or medical applications. In that regard, additives in soft tissue fillers can serve a number of applications, generally focused around patient comforts, filler a duration improvement, or some other clinical benefit.

Examples of such additives include topical anesthetics, vaso-constrictors, antioxidants, enzymatic degradation inhibitors, and antibiotics. Such additives are often desirably mixed with a filler formulation immediately prior to administration in order to provide a uniform and/or sustained delivery.

In certain cases, the filler and the additive cannot be mixed until a few minutes before injection. The two main reasons necessitating on-site mixing include stability concerns and a physician's option to choose an additive-filler combination immediately before injection. With regard to stability, the filler can have a negative effect on the stability of the additive or vice versa.

The present invention is directed to a dual-cartridge syringe designed to mix and dispense two fluids of interest.

### SUMMARY OF THE INVENTION

A dual cartridge mixer syringe in accordance with the present invention generally includes a first cartridge for containing a first formulation and a second cartridge for containing a second formulation.

A first plunger is provided for displacing the first formulation from the first cartridge and the second plunger is provided for displacing the second formulation from the second cartridge.

A mixing assembly is provided for receiving and commingling the first and second formulations in unequal portions. This is particularly important for use with a filler and an active agent.

A needle assembly is provided, which is in fluid communication with the mixing assembly, for injecting the commingled first and second formulations.

To provide for commingling the first and second formulations in unequal portions, the mixing assemble includes a manifold for separating a flow of the first formulation into a plurality of first fluid streams and merging the first fluid streams with a second fluid stream of the second formulation.

Particularly, the mixing assembly is configured for receiving the second fluid stream along a centerline thereof and applying the plurality of first fluid streams along a periphery of the second fluid stream. An insert may be provided for swirling together the first of fluid streams and the second fluid stream.

Preferably, the first cartridge has a smaller diameter than the second cartridge and the first and second plungers are interconnected by plunger handle for enabling simultaneous displacement of the first and second fluids. In addition, the mixing assembly is attachable to the first and second cartridges, thereby facilitating the disposal and the selection thereof by a position immediately before use.

### BRIEF DESCRIPTION OF THE DRAWINGS

The advantages and features of the present invention will be better understood by the following description when considered in conjunction with the accompanying drawings in which:

FIG. 1 is a perspective view of a dual cartridge mixer syringe in accordance with the present invention generally showing first and second cartridges along with a first and second plungers, a mixing assembly, and a needle assembly;

FIGS. 2 and 3 are perspective views of the mixing assembly in accordance with the present invention;

FIG. 4 is a separated and exploded view of the mixing assembly in accordance with the present invention further illustrating a swirling insert;

FIG. 5 is a cross sectional view of the mixing assembly illustrating its attachment to the first and second cartridges and needle assembly as well as detailing a manifold for separating flow of the first formulations; and

FIG. 6-8 illustrates operation of a dual cartridge mixing syringe in accordance with the present invention.

### DETAILED DESCRIPTION

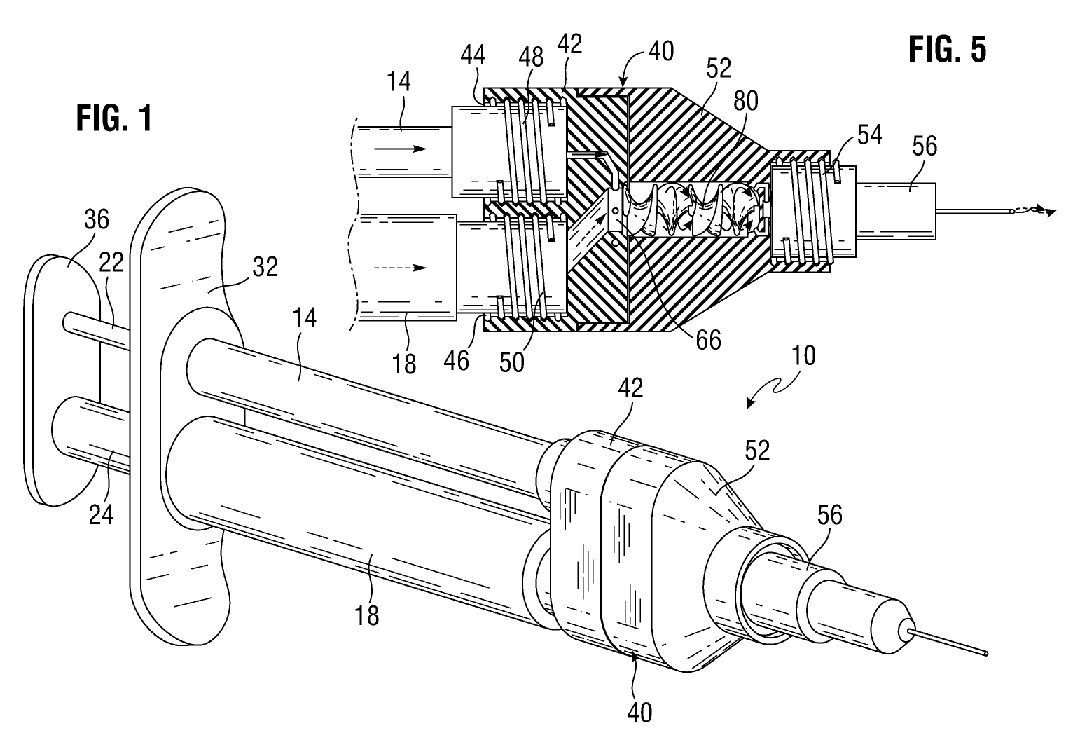

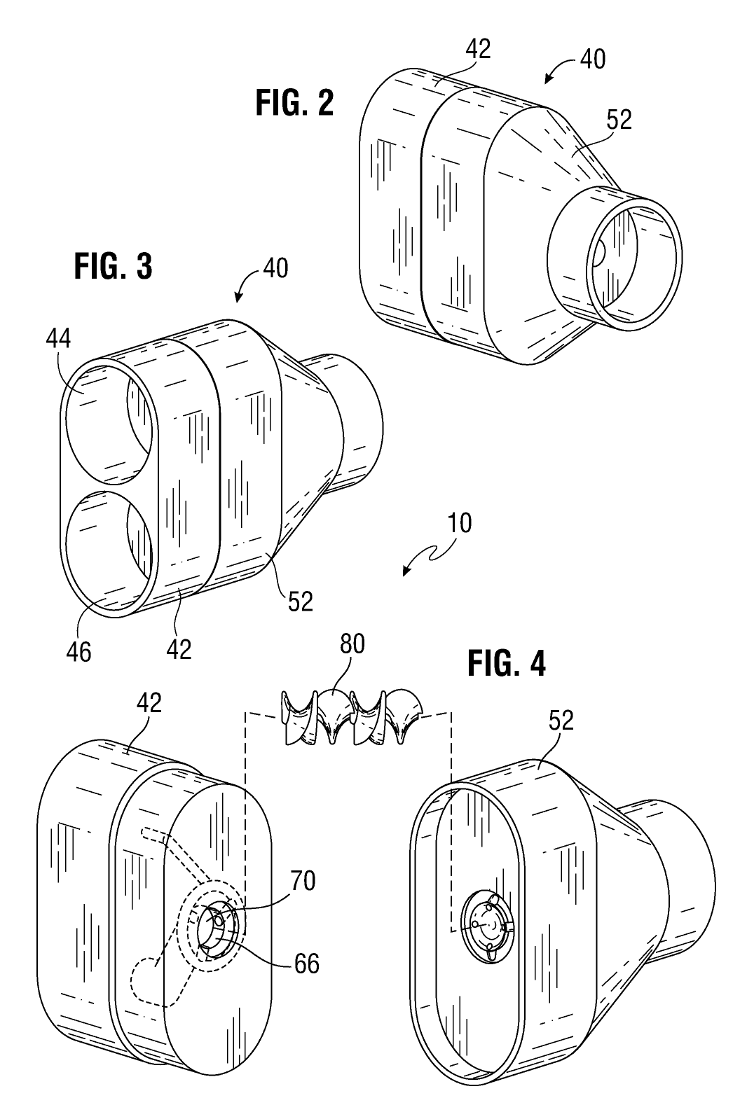

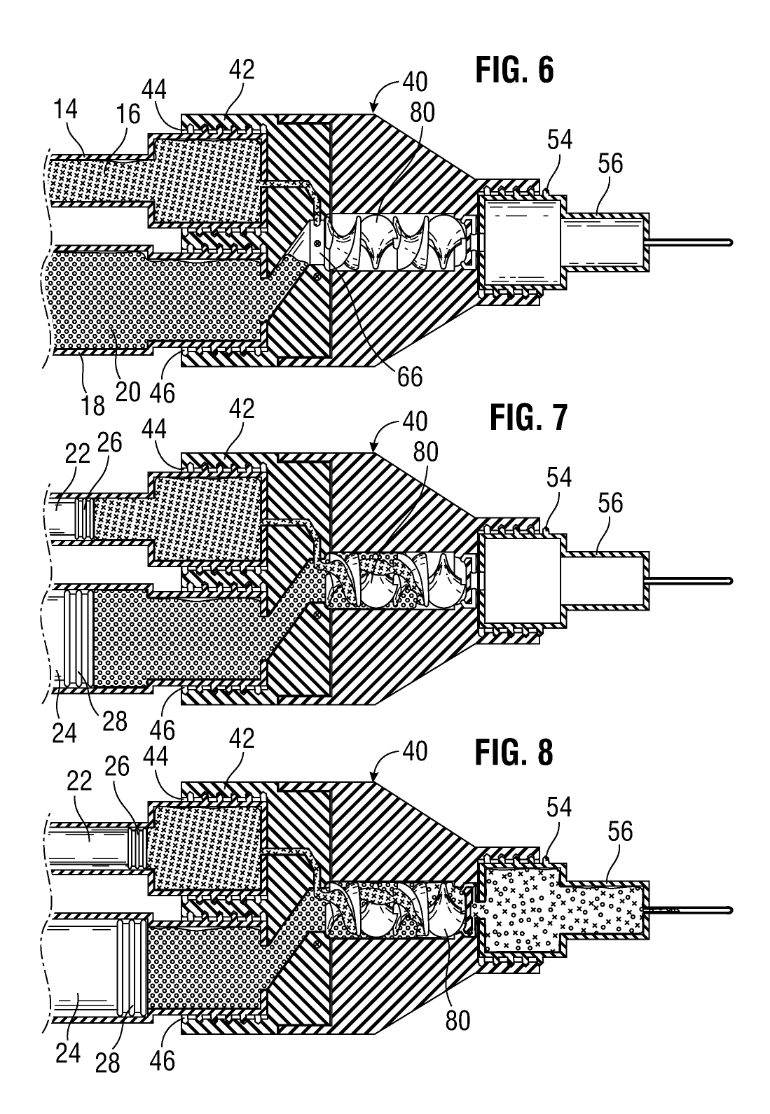

With reference to FIG. 1, there is shown a dual cartridge mixer syringe 10 in accordance with the present invention, which generally includes a first cartridge 14 for containing a first formulation 16 and a second cartridge 18 for containing a second formulation 20 (see FIGS. 6-8). The cartridges 14, 18 may be preloaded with formulations 16, 20 and, in that instance, become part of the present invention.

A first plunger 22 and a second plunger 24 include respectively plunger tips 26, 28 sized for slidable movement within the respective cartridges 14, 18 for displacing respectively first formulation 16 from the first cartridge 14 and second formulation 20 from the second cartridge 18.

A finger grip 32 supports and stabilizes the cartridges 14, 18 and facilitates the movement of the plungers 22, 24 by a plunger push button 36 encompassing both the first and second plungers 22, 24 for enabling simultaneous displacement of the first and second formulations 16, 20.

A mixing assembly 40 includes a base 42 with two entry ports 44, 46 which may be coupled with the first and second cartridges 14, 18 by way of thread connections 48, 50 as shown in FIG. 5. The mixing assembly 40 further includes a mixing tip 52 for threaded 54 engagement with a syringe needle 56. Alternative connecting mechanisms, such as a luer-lock or snap engagement, may be utilized.

The mixing assembly 40 provides for receiving and commingling the first and second formulations 16, 20 in unequal portions as follows:

As best shown in FIGS. 4-5, the mixing assembly base 42 includes a manifold 66 for separating a flow of the first formulation 16 into a plurality of first fluid streams and merging the first fluid streams with a second fluid stream of the second formulation 20 the latter being introduced along a central passage 70 of the manifold 66, see FIG. 4.

As illustrated in FIGS. 6-8, the manifold 66 and mixing assembly 40 provides for applying the plurality of first fluid streams along a periphery of the second fluid stream.

Further illustrated in FIGS. 1, 4-8, is an insert 80, which may be screw-like in configuration, for swirling together the first fluid stream and the second fluid stream due to movement of the first and second fluid streams therepast caused by displacement of the first and second fluid formulations from cartridges 14, 16 by the plungers 22, 24.

Preferably, the first cartridge 22 is a smaller diameter than the second cartridge 24 for further enabling commingling of the first and second formulations 16, 20 in unequal proportions The first formulation 16 may contain an active agent while the second formulation typically comprises a filler material.

It follows that a method in accordance with the present invention for dispensing a first and second formulation generally includes separating the first formulation into multiple streams within a syringe assembly along with simultaneously creating a single flow of the second formulation for ejection through a needle.

The method further provides for merging, the second formulation flow with the multiple streams of the first formulation with the multiple streams being applied to a periphery of the second formulation flow. Thereafter, the multiple streams are commingled for ejection through a needle as a homogeneous output of first and second formulations.

In that regard, the method may further utilize for a flow driven insert 80 within the syringe assembly for enhancing the commingling of the first and second fluids.

Although there has been hereinabove described a specific dual cartridge mixer syringe in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A dual cartridge mixer syringe comprising:
a first cartridge for containing a first formulation;
a second cartridge for containing a second formulation;
a first plunger for displacing said first formulation from said first cartridge;
a second plunger for displacing said second formulation from said second cartridge;
a mixing assembly for receiving and commingling the first and second formulation in unequaled proportions; and
a needle assembly, in fluid communication with said mixing assembly, for ejecting the commingled first and second formulation.

2. The syringe according to claim 1 wherein said mixing assembly comprises a manifold for separating a flow of said first formulation into a plurality of first fluid streams and merging the first fluid streams with a second fluid stream of said second formulation.

3. The syringe according to claim 2 wherein said mixing assembly is configured for receiving the second fluid stream along a centerline thereof and applying the plurality of first fluid streams along a periphery of the second fluid stream.

4. The syringe according to claim 3 wherein said mixing assembly further comprises an insert for swirling together the first fluid streams and the second fluid stream.

5. The syringe according to claim 1 wherein said first cartridge has a smaller diameter than said second cartridge.

6. The syringe according to claim 5 wherein the first and second plunger are interconnected by a plunger push button for enabling simultaneous displacement of the first and second formulation.

7. The syringe according to claim 6 wherein said mixing assembly comprises a manifold for separating a flow of said first formulation into a plurality of first fluid streams and merging the first fluid streams with a second fluid stream of said second formulation.

8. The syringe according to claim 7 wherein said mixing assembly is configured for receiving the second fluid stream along a centerline thereof and applying the plurality of first fluid streams along a periphery of the second fluid stream.

9. The syringe according to claim 8 wherein said mixing assembly further comprises an insert for swirling together the first fluid streams and the second fluid stream.

10. A dual cartridge mixer syringe comprising:
a first cartridge containing a first formulation;
a second cartridge containing a second formulation;
a first plunger for displacing said first formulation from said first cartridge;
a second plunger for displacing said second formulation from said second cartridge;
a mixing assembly, attachable to the first and second cartridges, for receiving and commingling the first and second formulations in unequal proportions; and
a needle assembly attachable to said mixing assembly for fluid communication therewith for ejecting the commingled first and second formulations.

11. The syringe according to claim 10 wherein said mixing assembly comprises a manifold for separating a flow of said first formulation into a plurality of first fluid streams and merging the first fluid streams with a second fluid stream of said second formulation.

12. The syringe according to claim 11 wherein said mixing assembly is configured for receiving the second fluid stream along a centerline thereof and applying the plurality of first fluid streams along a periphery of the second fluid stream.

13. The syringe according to claim 12 wherein said mixing assembly further comprises an insert for swirling together the first fluid streams and the second fluid stream.

14. The syringe according to claim 10 wherein said first cartridge has a smaller diameter than said second cartridge.

15. The syringe according to claim 14 wherein the first and second plunger are interconnected by a plunger push button for enabling simultaneous displacement of the first and second formulation.

16. The syringe according to claim 15 wherein said mixing assembly comprises a manifold for separating a flow of said first formulation into a plurality of first fluid streams and merging the first fluid streams with a second fluid stream of said second formulation.

17. The syringe according to claim 16 wherein said mixing assembly is configured for receiving the second fluid stream along a centerline thereof and applying the plurality of first fluid streams along a periphery of the second fluid stream.

18. The syringe according to claim 17 wherein said mixing assembly further comprises an insert for swirling together the first fluid streams and the second fluid stream.

19. A method for dispensing a first and second formulation comprising:
a separating the first formulation into multiple streams within a syringe assembly;
creating a flow of the second formulation for ejecting through a needle;
merging the second formulation flow with the multiple streams of first formulation, the multiple stream being applied to a periphery of the second formulation flow; and
commingling the multiple streams and second formulation flow before ejecting through said needle.

20. The method according to claim 19 further providing a flow driven insert within said syringe assembly for commingling of the first and second formulation.
